(19) United States
(12) Patent Application Publication
(10) Pub. No.: US 2003/0236543 A1
(43) Pub. Date: Dec. 25, 2003

(54) Polygonal cross section lancet needle

(76) Inventors: Allen J. Brenneman, Goshen, IN (US); Mohammad A. Kheiri, Elkhart, IN (US)

Correspondence Address: Jerome L. Jeffers, Esq., Bayer Healthcare, LLC, P. O. Box 40, Elkhart, IN, 46515-0040, US

(21) Appl. No.: 10/448,494

(22) Filed: Jun. 2, 2003

Related U.S. Application Data
(60) Provisional application No. 60/390,893, filed on Jun. 25, 2002.

(51) Int. Cl. A61B 17/34

(52) U.S. Cl. 606/181

(57) ABSTRACT

A lancet needle for lancing finger tips and other sites on a body to draw small samples of blood has a multisided body. The body has one end that has a portion at an angle to define a sharp edge adapted to lance a finger tip or other body site.

### FIELD OF THE INVENTION

The present invention relates generally to a lancet needle for lancing fingers and other body sites to obtain small samples of blood or interstitial fluid for diagnostic testing. More particularly, the invention relates to a lancet needle having a polygonal or multisided cross section.

### BACKGROUND OF THE INVENTION

People with certain medical conditions, such as diabetics, often have to perform diagnostic tests on their blood for glucose and other analytes several times a day. To performn these tests, a site on the body is selected and lanced using a lancet assembly that includes a lancet needle. These lancet needles are made from round stainless steel wires with tips that are ground to form two or three facets on the tips. Round needles force the skin open after initial penetration and only cut at the ground facets which results in a large puncture that is painful and requires a long time to heal.

Recently, instruments for performing diagnostic tests have been developed that require much smaller samples of blood or interstitial fluid than the sample size required by earlier instruments. In addition, patients have demanded less painful lancing methods. Thus, there is a need for lancing needles whose puncture is less painful than current needles and produce a small wound that is quick to heal.

### SUMMARY OF THE INVENTION

The present invention is directed to a multisided lancet needle that makes a small, quick healing cut at a puncture site. The lancet needle is defined by a body having a polygonal cross section and a first end and a second end. The first end is ground at an angle, for example, an angle between about 5° and about 10°, to form a sharp edge for lancing the skin of a user. The cross section of the needle body can be square, triangular, rectangular, or any similar multisided configuration.

### BRIEF DESCRIPTION OF THE FIGURES

Other objects and advantages of the invention will become apparent upon reading the following detailed description and upon reference to the drawings in which:

FIG. 1 is a perspective view of a square lancet needle with a single cut;

FIG. 2 is a perspective view of a square lancet needle with a double cut;

FIG. 3 is a perspective view of a square lancet needle with a diagonal cut;

FIG. 4 is a perspective view of a triangular lancet needle with a single cut;

FIG. 5 is a perspective view of a rectangular lancet needle with a single cut;

FIG. 6 is a plan view of a round wire with one end ground to a preferred configuration;

FIG. 7 is a view of the wire illustrated in FIG. 6 with the one end ground to define a sharp edge; and

FIG. 8 is an enlarged view of a square lancet needle with a tip ground at an angle.

While the invention is susceptible to various modifications and alternative forms, a specific embodiment thereof has been shown by way of example in the drawings and will herein be described in detail. It should be understood, however, that it is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF THE ILLUSTRATED EMBODIMENTS

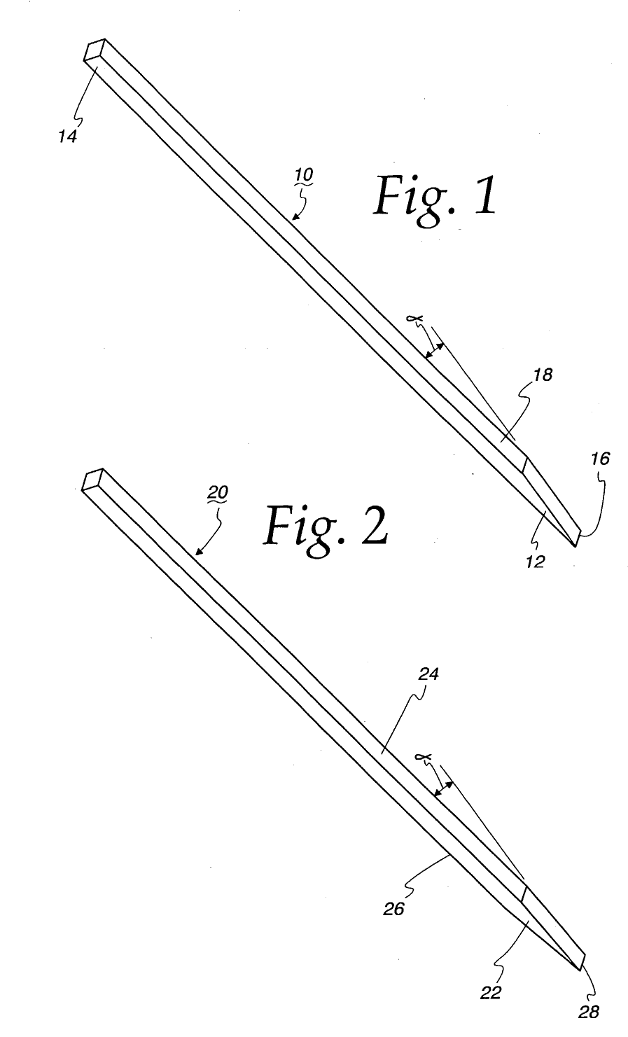

Referring to FIG. 1, there is illustrated a lancet needle 10 having a square cross section. The lancet needle 10 is formed of stainless steel but other material may be used. The lancet needle 10 has a first end 12 and a second end 14. The first end 12 has a sharp edge or tip 16 formed by grinding one side 18 of the lancet needle 10 on an angle ∀ to the one side 18. Preferably, the angle ∀ is in the range of between about 5° to about 10° but other angles may be used. It has been learned that this angle ∀ in the range of about 5° to about 10° provides a sufficiently sharp edge or tip 16 to penetrate and cut human skin and tissue in a very clean way with less pain to the user than the pain experienced using prior art round needles.

Another lancet needle 20 of square cross sectional configuration is shown in FIG. 2. This lancet needle 20 is substantially the same as the lancet needle 10 of FIG. 1 except that its first end 22 is ground on opposite sides 24, 26 to form a sharp edge or tip 28. Each side 24, 26 is ground at an angle 3 which preferably is in the range of about 5° to about 10° although other angles may be used.

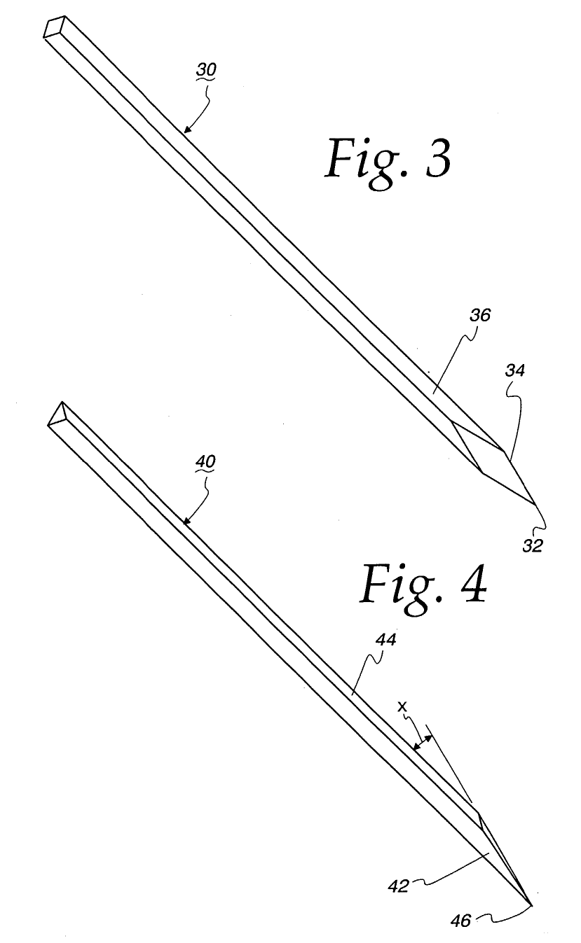

A third lancet needle 30 of square cross sectional configuration is illustrated in FIG. 3. The lancet needle 30 is ground on a diagonal by a diagonal cut to form a sharp edge or tip 32 on a first end 34 of the lancet needle 30. The first end 34 is ground diagonally 45° from a first side 36 at an angle in the range of about 5° to about 10° although other angles may be used.

A lancet needle 40 with a triangular cross sectional configuration is shown in FIG. 4. A first end 42 of the lancet needle 40 is ground on a side 44 at an angle X in the range of between about 5° and about 10° to form a sharp chisel point or edge 46.

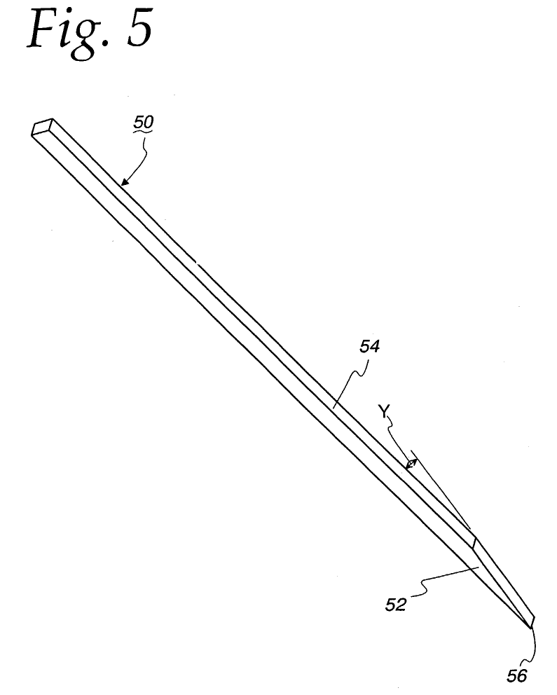

In FIG. 5 there is illustrated a lancet needle 50 having a rectangular cross sectional configuration. A first end 52 of the lancet needle 50 has one side 54 ground at angle Y that is in the range of between about 5° to about 10° to define a sharp edge 56 for piercing skin and tissue.

The sharp edges or tips 16, 28, 32, 46, 56 of the needles 10, 20, 30, 40, 50 penetrate a user's skin and continue to cut the skin and tissue in a clean way resulting in a lower pain level and a cut or puncture that heals faster than a cut from a conventional round lancet needle. This is because the multisided cross sections cut on all sides and do not force skin open as much as round needles. Thus, multisided configurations such as hexagonal are also contemplated.

Manufacturing these lancet needles 10, 20, 30, 40, 50 is similar to the manufacture of conventional round needles. Multisided cross sectional wires are extruded from a round stainless steel wire and cut into short workable lengths for grinding. The tips or first ends of a large number of these wires are ground to the desired angle at the same time. The needles are then cleaned and deburred before being molded in plastic to form a lancet. The molded lancets are then sterilized by radiation.

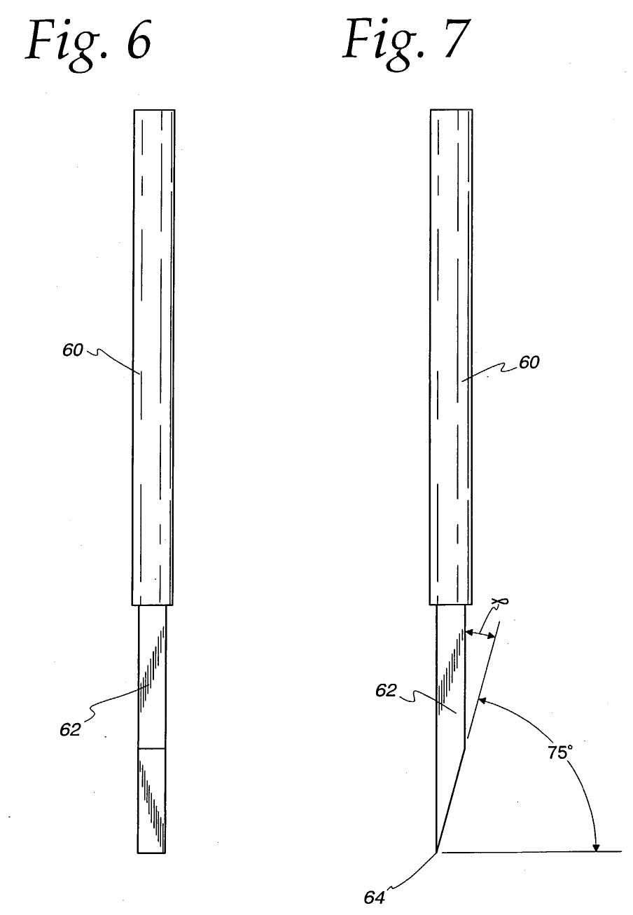

An alternative method of manufacture can be envisioned which will require less change to the manufacturing of conventional round needles. Rather than extruding round wire to a different cross-section, a conventional round wire 60 is ground on a first end 62 to a preferred multisided cross section (e.g., square, triangular, rectangular, etc.) (FIG. 6). The first end 62 is then ground at an angle from about 5° to about 10° to form a sharp edge or tip 64 (FIG. 7). At this point the ground wire 60 can be insert molded into plastic. This method allows the use of large diameter round wires 60, 22-26 gauge for example, since the first end 62 can be ground to a small cross section profile (29-32 gauge for example). The larger gauge is easier to handle for manufacturing and has more surface area to lock into molded plastic. This results in the same cutting edges as in the previously described lancets. In addition the step formed at the transition from the round to the smaller cross section will also provide a means of locking the needle into the plastic.

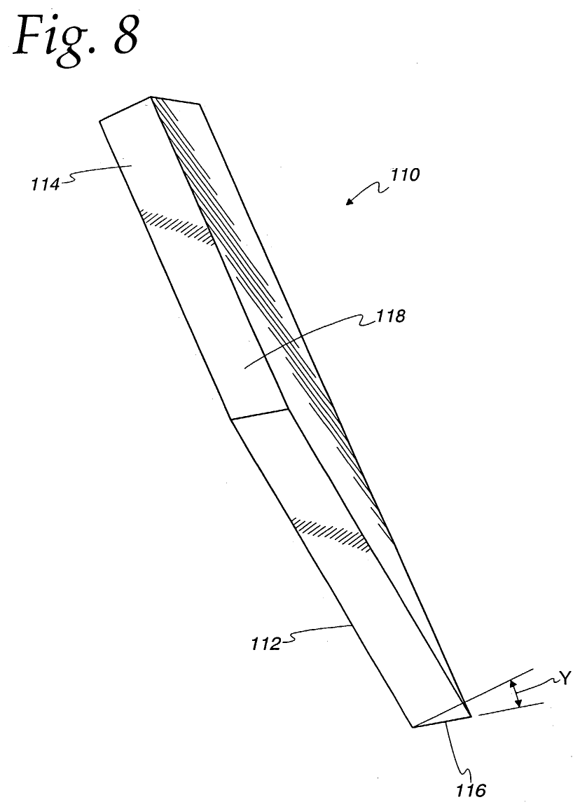

It has been found that grinding an end of a needle at an angle to the longitudinal axis of the needle results in an angle at the tip which can start a cut in skin at an end of the tip rather than a cut being started along the entire length of the tip. This causes less pain to the user or patient. This angled grind is shown in FIG. 8. A lancet needle 110 may be square or rectangular with a first end 112 and a second end 114. The needle 110 can be ground on one side 118 or a double grind as in FIG. 2 but the grind in FIG. 8 is at an angle to the longitudinal axis of the needle 110 resulting in an angled tip 116 at an angle Y in the range of 0° to 15°.

While the present invention has been described with reference to one or more particular embodiments, those skilled in the art will recognize that many changes may be made thereto without departing from the spirit and scope of the present invention. Each of these embodiments and obvious variations thereof is contemplated as falling within the spirit and scope of the claimed invention, which is set forth in the following claims.

What is claimed is:

1. A needle for a lancet assembly, comprising:
a needle body, said needle body being of a multiside configuration including a first end and a second end, said first end including a sharp edge.

2. The needle claimed in claim 1 wherein the cross section of said needle body is square.

3. The needle claimed in claim 1 wherein the cross section of said needle body is triangular.

4. The needle claimed in claim 1 wherein a portion of said first end is at an angle between about 5° and about 10° to define said sharp edge.

5. The needle claimed in claim 1 wherein the cross section of said needle body is rectangular.

6. The needle claimed in claim 1 wherein said body is square, one side of said square being at an angle between about 5° to about 10° to define said sharp edge.

7. The needle claimed in claim 1 wherein said body is in the configuration of a triangle with a portion of said triangle being at an angle of between about 5° and about 10° to define said sharp edge.

8. A lancet needle, comprising:
a needle body, said needle body including a plurality of sides, said needle body including a first end and a second end, a portion of one of said sides adjacent said first end being at a first angle to define a sharp edge at said first end.

9. The lancet needle claimed in claim 8 wherein said needle body has a square cross sectional configuration.

10. The lancet needle claimed in claim 8 wherein said needle body has a triangular cross sectional configuration.

11. The lancet needle claimed in claim 8 wherein said needle body has a rectangular cross sectional configuration.

12. The lancet needle claimed in claim 8 wherein said first angle is between about 50 and about 10°.

13. The lancet needle claimed in claim 8 including a second angle in a portion of another of said sides adjacent said first end, said first angle and said second angle defining said sharp edge.

14. A method of making a multisided lancet needle, comprising:
providing a multisided body having a first end and a second end, and forming an angle in said first end to define a sharp point.

15. The method claimed in claim 14 wherein said step of providing a multisided body comprises providing a four sided body.

16. The method claimed in claim 14 wherein said step of providing a multisided body comprises providing a three sided body.

17. The method claimed in claim 14 wherein said step of forming an angle comprises forming an angle between from about 5° to about 10°.

18. A method of making a multisided lancet needle, comprising:
providing a round wire having a first end,
grinding at least a portion of said first end into a multisided configuration, and
grinding said first end at an angle from about 5° to about 10° to form a sharp edge.

19. A needle for a lancet assembly, comprising:
a multisided needle body, said needle body including a first end and a second end, said first end including a sharp edge, said sharp edge being at an angle to the longitudinal axis of said multisided body.

20. The needle claimed in claim 19 wherein said angle is between 0° and 15°.
